(19) United States
(12) Patent Application Publication
(10) Pub. No.: US 2006/0247766 A1
(43) Pub. Date: Nov. 2, 2006

(54) System for enlarging a retinal image

(76) Inventor: Gildas Marin, Antony (FR)

Correspondence Address: FISH & RICHARDSON PC, P.O. BOX 1022, MINNEAPOLIS, MN, 55440-1022, US

(21) Appl. No.: 11/404,491

(22) Filed: Apr. 13, 2006

Related U.S. Application Data
(63) Continuation of application No. PCT/FR04/02581, filed on Oct. 12, 2004.

(30) Foreign Application Priority Data
Oct. 14, 2003 (FR) 03 12 009

(51) Int. Cl. A61F 2/16

(52) U.S. Cl. 623/6.11

(57) ABSTRACT

The invention relates to a system for enlarging a retinal image, comprising an intraocular implant and an external lens. The implant comprises a peripheral part and a central part with a negative power. The lens has a positive power and is for arrangement outside the eye, typically in a glasses frame. The lens and the implant produce an enlarged image of an object at the back of the eye of a standard user. For a pupil with a diameter of 1.5 mm, each point object in a reading field forms a dark image of a size between 20 and 50 μm at the back of the eye. The invention permits a large reading field, at the cost of a degradation in the performance of the system along the axis, acceptable when taking into account the acuity of the patients treated and, furthermore, provides a system with little variation in performance with displacement of the lens from the nominal position.

### CROSS REFERENCE TO RELATED APPLICATIONS

This application is a continuation of, and claims the benefit of, international application number PCT/FR2004/002581, filed Oct. 12, 2004, which designates the United States and was published as PCT publication number WO 2005/037145, which in turn claims priority to French application number 0312009, filed Oct. 14, 2003. These applications are incorporated herein by reference.

### BACKGROUND OF THE INVENTION

This invention relates to systems for enlarging a retinal image, used for optical correction of macular degeneration.

Age-related macular degeneration (ARMD) is a disorder of the macula, which extends over the top of the retina at the back of the eye. This degeneration corresponds to a loss of the activity of the retinal rods situated in the macula and causes the affected person to lose a large part of his visual acuity. In near vision, the affected person loses the capacity to read; in distance vision, walking becomes a difficult activity. Currently no treatment exists that allows for this degeneration to be cured. Only visual aids providing a magnifying power allow for partial compensation for the affected person's drop in acuity in near vision to the detriment of a more reduced field. However, for distance vision, which is often used when the person moves, the field must be wide and the magnification close to 1 in order not to impede the wearer's perception of space. Devices for compensation for ARMD must therefore have two distinct operating states: with and without magnification.

A cataract causes partial or total opacity of the crystalline lens. A cataract is in particular treated by replacing the crystalline lens with an ocular implant, commonly called an intrasaccular implant due to its positioning in the capsular sac.

In order to provide the magnification used for the correction of ARMD, U.S. Pat. No. 4,957,506 discloses a system made up of a lens with strong positive power, designed to be placed in front of the eye, and an intraocular implant with strong negative power, replacing the crystalline lens. At least one surface of the lens and of the implant is aspherical. The system does not provide adequate vision in the absence of the lens with strong positive power.

U.S. Pat. No. 4,666,446 discloses an intraocular implant for patients affected by macular degeneration, designed to replace the crystalline lens. The implant has a first diverging portion and a second converging portion, superimposed or concentric in the figures. The converging portion provides the patient with vision more or less identical to that which he had before the replacement of the crystalline lens by the implant, in other words vision without magnification. The diverging portion, when it is combined with a lens external to the eye, forms a telescopic system and provides an enlarged image of a given object.

U.S. Pat. No. 4,932,971 discloses a solution for the treatment of patients affected by macular degeneration, who already have an intrasaccular implant. This document describes a lens provided with extensions, which attach to the peripheral portion of an intrasaccular implant. The lens in this document can thus be attached in situ onto a lens implant, implanted beforehand, without the need to remove the implant or to provide haptics other than those of the existing implant.

U.S. Pat. No. 6,197,057 also discloses a system for the correction of macular degeneration. This system uses an intraocular implant, which is placed in the eye in front of the crystalline lens or in front of an intrasaccular implant replacing the crystalline lens. In one embodiment, the intraocular implant has a central zone with strong negative power and a peripheral zone with no refractive effect on the light that passes through it. In this embodiment the system provides normal vision in the absence of a lens external to the eye; in the presence of an external lens with strong positive power the system provides an enlarged image. The principle of correction is therefore similar to that described in U.S. Pat. No. 4,666,446. In a second embodiment, the intraocular implant is prism-shaped and the effect of the system is to redirect the rays entering the eye towards a portion of the retina other than the macula, which is not affected by macular degeneration.

WO-A-93 01765 and U.S. Pat. No. 5,030,231 describe other retinal image magnification systems. These documents provide no indication as to the size of the image spot outside the axis of the system.

U.S. Pat. No. 5,532,770 describes a method and a device allowing for the evaluation of a subject's vision through an intraocular implant. It is stated that different positions of the implant in the eye can be considered. However, it is not suggested in this document that the different positions are different positions of the same implant, nor that the modifications of the position of the implant can be taken into account in the same subject. On the contrary, this document mentions different implants, or different positions of the implant.

In a retinal image magnification system it is advantageous to have as large a field of vision as possible. In particular, the field of vision should make it possible to read easily.

Another newly identified problem of the systems of the state of the art is that they are sensitive to incorrect positioning. The different components of the telescopic system—lens external to the eye and implant—have strong power. The decentering or angular displacement (tilt) of the elements of the telescopic system can considerably reduce the field of vision and the characteristics of the system. This is all the more problematic as the implantation cannot guarantee very precise positioning: regardless of the precision of implantation, the tissue grows after the operation and can lead to displacement of the implant.

Moreover, the system is designed for patients with visual impairment affected by macular degeneration; these patients have often lost their capacity to fix on an object, as a result of the loss of central vision, and generally use their peripheral vision without it being possible to guarantee that their eccentric direction of viewing is stable. The age of the patient can also make it difficult to take measurements for precise positioning of the external lens.

### SUMMARY OF THE INVENTION

The invention provides a solution to one or more of these problems of the state of the art. It provides, in one embodiment, a system for enlarging a retinal image, comprising:

an intraocular implant having a peripheral portion and a central portion with negative power,

a lens with positive power designed to be arranged outside the eye,

the lens and the implant designed to produce an enlarged image of an object at the back of the eye of a standard user,

in which, for a pupil 1.5 mm in diameter, any point object in a reading object field produces at the back of the eye an image spot of a size comprised between 20 and 50 μm, for a wavelength in the visible spectrum.

Advantageously, when the angular position of the lens varies in a range of ±2° relative to its nominal position, for a pupil 1.5 mm in diameter, any point object in a reading object field produces at the back of the eye an image spot of a size comprised between 20 and 50 μm, for a wavelength in the visible spectrum. It is also possible to set this condition for a variation in a range of ±5°, or in a range of ±10°.

Preferably, when the decentering of the lens varies in a range of ±0.2 mm relative to the nominal position, any point object in a reading object field produces at the back of the eye an image spot of a size comprised between 20 and 50 μm, for a wavelength in the visible spectrum. It is also possible to set this condition for a variation in a range of ±1 mm, or in a range of ±2 mm.

In one embodiment, the lens has diffractive properties, for example obtained by modification of the profile of one of the surfaces of the lens. In this case, it is advantageous that when the decentering of the lens varies in a range of ±1 mm, relative to the nominal position, any point object in a reading object field produces at the back of the eye an image spot of a size comprised between 5 and 80 μm, for three wavelengths distributed in the visible spectrum.

It can also be anticipated that, when the angular position of the lens varies in a range of ±5°relative to its nominal position, for a pupil 1.5 mm in diameter, any point object in a reading object field produces at the back of the eye an image spot of a size comprised between 5 and 80 μm, for three wavelengths distributed in the visible spectrum.

The three wavelengths distributed in the visible spectrum can be respectively chosen in the ranges of 400 to 500 nm, 500 to 600 nm and 600 to 800 nm.

The system can also have one or more of the following characteristics:

the lens is a Fresnel lens;

the central portion of the implant is spherical;

the front face of the lens is a cone the conicity of which is comprised between 0 and −1, and preferably comprised between −0.2 and −0.6;

the system has a magnification comprised between 2 and 4;

the system has, in the conditions of use, a distance between the lens and the implant greater than or equal to 19 mm;

the reading object field is situated at a distance of 25 cm from the lens and covers an angle of 10°;

the reading object field is defined by an aperture angle at the retina of ±24°.

The invention also provides, in another embodiment, a method for determination by optimization of a system for enlarging a retinal image, comprising:

choosing an eye model, wearing conditions, an intraocular implant and a lens external to the eye;

modifying the characteristics of the implant and of the lens in order that, in a reading object field, any point object produces at the back of the eye an image spot of a size comprised between 20 and 50 μm, for a wavelength in the visible spectrum.

Advantageously, the modification stage is also carried out in order that, in the presence of a variation of the angular position of the lens relative to the chosen wearing conditions, in a range of ±2°, any point object in a reading object field produces at the back of the eye an image spot of a size comprised between 20 and 50 μm, for a wavelength in the visible spectrum. It is also possible to set this limit for a variation of the angular position of the lens in a range of ±5°, or in a range of ±10°.

It is also possible for the modification stage to be carried out in order that, in the presence of a decentering of the lens in a range of ±0.5 mm relative to the chosen wearing conditions, any point object in a reading object field produces at the back of the eye an image spot of a size comprised between 20 and 50 μm, for a wavelength in the visible spectrum. It is also possible to set this limit for a decentering of the lens in a range of ±1 mm, or ±2 mm.

It is also possible to provide for the modification stage to comprise the application of diffractive properties to the lens. In this case, the modification stage can be carried out in order that, in the presence of a variation of the angular position of the lens relative to the chosen wearing conditions, in a range of ±5°, any point object in a reading object field produces at the back of the eye an image spot of a size comprised between 5 and 80 μm, for three wavelengths distributed in the visible spectrum.

It is also possible to provide for the modification stage to be carried out in order that, in the presence of a decentering of the lens in a range of ±1 mm relative to the chosen wearing conditions, any point object in a reading object field produces at the back of the eye an image spot of a size comprised between 5 and 80 μm, for three wavelengths distributed in the visible spectrum.

The object field can be defined in the method as situated at a distance of 25 cm from the lens and covering an angle of 10°, or can also be defined by an aperture angle at the retina of ±24°.

Other advantages and characteristics of the invention will become apparent when reading the following description of embodiments of the invention, given as an example and with reference to the drawings.

### BRIEF DESCRIPTION OF THE DRAWINGS

FIG. 1 is a cross-sectional top view of an eye-lens optical system with an implant according to the invention;

FIG. 2 is a larger-scale vertical cross-sectional view of the eye-lens system;

FIG. 3 is a graph of the distance of the reading object field as a function of the lens-eye distance in a system according to the invention and according to the state of the art;

FIG. 4 is a graph of the size of the image spot in the object field of a system according to the invention compared with the image spot of a system according to the state of the art;

FIG. 5 is a graph corresponding to the graph in FIG. 4, with a decentering of the lens of 1 mm;

FIG. 6 is a graph corresponding to the graph in FIG. 4, with an angular displacement of the lens of 5°;

FIG. 7 is a view similar to the view in FIG. 1 in an embodiment of the invention using a Fresnel lens;

FIGS. 8 to 10 are graphs similar to those in FIGS. 4 to 6, but for a third embodiment of the invention;

FIG. 11 is a graph similar to the graph in FIG. 8, but taking into account several wavelengths in the visible spectrum.

### DETAILED DESCRIPTION OF PREFERRED EMBODIMENTS

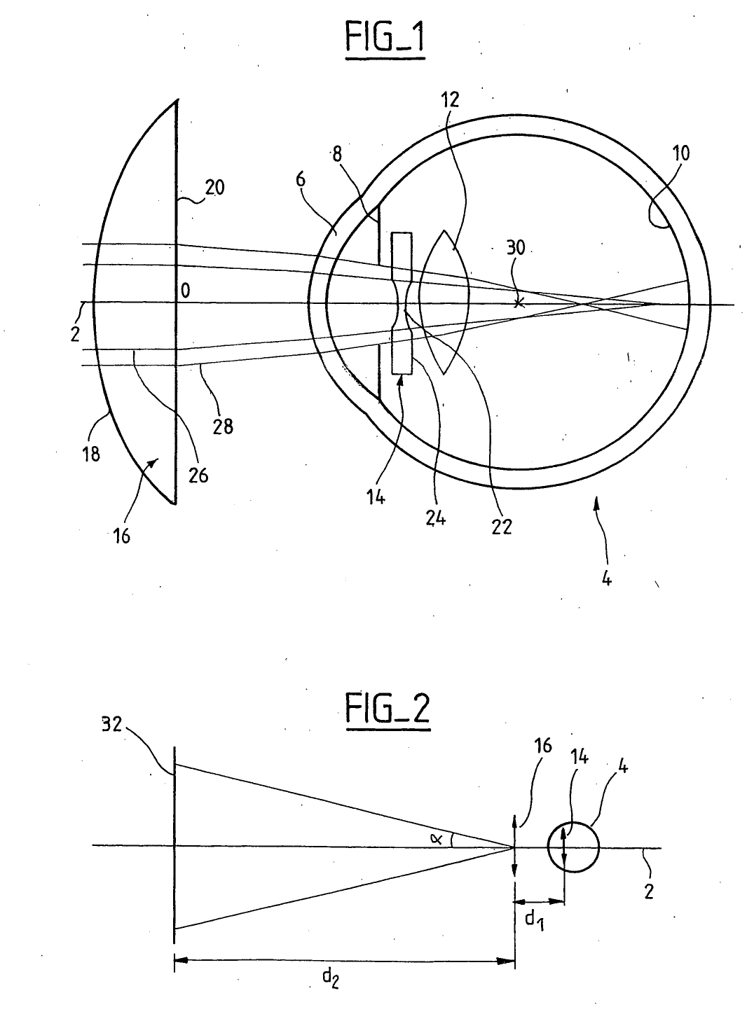

FIG. 1 shows a diagram of an eye-lens optical system according to the invention. The lens external to the eye is referred to in the following simply as a “lens”; likewise, the intraocular implant is designated simply by the term “implant” in the rest of the description. The lens and the implant produce a enlarging the image projected onto the back of the eye, in the manner of a telescope. The lens-implant assembly is therefore referred to in the following as a “telescopic system”, even though, strictly speaking, it is not a telescope.

In the figure an axis 2 corresponding to the primary direction of viewing is shown. The axis 2 passes through the center of rotation 30 of the eye 4. The eye is represented schematically; the cornea 6, the pupil 8, the retina 10, the crystalline lens or the intrasaccular implant 12 as well as an intraocular implant 14 according to the invention can be seen. The model proposed in Accommodation-dependent model of the human eye with aspherics, R. Navarro, J. Santamaria and J. Bescos, Vol. 2, No. 8/August 1985, Opt. Soc. Am. A. can be used as the eye model replacing, if appropriate, the crystalline lens with an intrasaccular implant.

The figure also shows the lens 16 external to the eye. The lens is mounted in a spectacle frame, in front of the eye.

The axis 2 cuts through the front face 18 of the lens, at a point which is generally situated 4 mm above the geometric center of the front face, when the lens is used both for distance vision and near vision and for a standard positioning of the frame. In the case of a telescopic system according to the invention, the lens is used only for near vision and it is advantageous that the axis 2 cuts through the front face 18 directly at its geometric center. Let point O be the point of intersection of the rear face and the axis 2. In a vertical plane containing the axis 2, the tangent to the rear face 20 of the lens at point O forms with a vertical axis passing through the point O an angle known as the pantoscopic angle. In the horizontal plane containing the axis 2, which is shown in the figure, the tangent to the rear face of the lens at the point O forms with an axis orthogonal to the axis 2 an angle called the curving contour. The term “wearing conditions” refers to the values of the distance between the point O and the center of rotation of the eye, the pantoscopic angle and the curving contour. For the wearing conditions it is possible to choose a triplet corresponding to mean values. It is also possible to vary the values for each individual or type of case. In the example in FIG. 1, it can be seen that the rear face is flat and that the curving contour is nil.

The choice of wearing conditions and of an eye model allows for complete modelling of the effects of an external lens and an implant according to the invention. In the case of a telescopic system according to the invention, the lens is only used for near vision and it is advantageous that the pantoscopic angle and the curving contour are nil.

If appropriate, it is possible to replace the crystalline lens with an intrasaccular implant and take into account the characteristics of the intrasaccular implant. It is simpler to place an implant behind the pupil, as shown in FIG. 1, when the crystalline lens is or has been replaced with an intrasaccular implant. Such an intrasaccular implant has a thickness of the order of one millimetre, which is less than the thickness of a natural crystalline lens, which is of the order of 4 millimetres. It can however be possible to arrange an implant with the natural crystalline lens, in the configuration shown in FIG. 1. An implant arranged in front of the pupil, in combination with a natural crystalline lens, could also be used, which would avoid any problem that the thickness of the crystalline lens might pose. FIG. 1 does not show the attachment of the implant. It is possible to use haptics, in a manner known per se; it is also possible to use the solution proposed in U.S. Pat. No. 4,932,971, and attach the implant to an intrasaccular implant, implanted beforehand or at the same time.

The intraocular implant 14 has a central zone 22 having negative power, and a peripheral zone 24. The central zone typically has a diameter comprised between 1.5 and 2 mm. The peripheral zone can have a refractive power of nil. As explained below, it can also be used to correct residual ametropia of the patient.

It could equally be envisaged that the implant according to the invention purely and simply replaces the crystalline lens or the lens implant as in U.S. Pat. No. 4,666,446, in which case the peripheral zone 24 of the implant will have a positive power so as to compensate for the crystalline lens. The implant can then be positioned either in the anterior chamber or in the sac.

FIG. 1 schematically shows the focussed rays 26 passing through the lens 16, the aperture of the pupil 8 and the central zone 22 of the implant. These rays participate in the formation on the retina of an enlarged image. FIG. 1 also shows the rays 28, passing through the aperture of the pupil 8 but crossing the peripheral zone 24 of the implant, these rays diverging and not participating in the formation of an image on the retina.

The invention proposes to define the characteristics of the intraocular implant 14 and of the lens 16 taking into account possible variations of position of the lens relative to the nominal position of the lens in the system. It is based on the recognition that patients suffering from macular degeneration no longer have acuity in central vision and generally have only poor residual acuity—less than 2/10^{th}—due to their peripheral vision. It is therefore not necessary for the image spot produced by the implant in the eye, in the presence of the external lens, to be a dot. Compared to the telescopic systems of the state of the art, an acceptable reduction of the optical quality of the system at the center of the object field allows for improvement of the optical quality of the system at the periphery of the object field, or acceptance of the variations of the position of the lens relative to its nominal position.

The invention is based on the recognition that in the type of telescopic system in question, the field of vision is very quickly limited by the optical quality of the system if the lens and the intraocular implant are not simultaneously and correctly optimized, and this is not disclosed by U.S. Pat. No. 4,666,446, U.S. Pat. No. 4,932,971 and U.S. Pat. No. 6,197,057. U.S. Pat. No. 4,957,506 seeks to obtain very high optical quality, so that the system remains limited in the field of vision. This type of system is designed for patients with visual impairment affected by macular degeneration whose visual acuity is greatly reduced and who therefore do not require very good optical quality at the center of the field of vision. This characteristic is advantageously used in the invention to enlarge the field of vision.

FIG. 2 shows a larger-scale vertical cross-sectional schematic view of the eye-lens system. FIG. 2 shows the axis 2 of the principal viewing direction, the eye 4 with a schematic representation of the implant 14, a schematic representation of the lens 16, as well as the object field 32. d₁ denotes the distance between the front face of the implant and the rear face of the lens and d₂ the distance between the object and the front face of the lens. In the following examples, the eye model described in the article by R. Navarro et al. is considered.

For the wearing conditions a distance d₁ of 22.43 mm is considered. This distance corresponds, in the above-mentioned eye model, to a distance between the rear face of the lens and the eye of the order of 18 mm. This distance is greater than the usual distance considered for the wearing conditions, which is of the order of 27 mm for the distance between the rear face of the lens and the center of rotation of the eye, i.e. a distance of the order of 12 mm between the lens and the eye. At constant magnification, the fact of considering for the distance d₁ a value slightly higher than the usual value allows for a reduction of the power of the lens and the implant. The tolerances of the telescopic system are improved relative to the shortcomings in positioning of the lens. It is therefore advantageous for the wearing conditions considered to use a distance between the lens and the center of rotation of the eye of the order of 33 mm, or a distance between the lens and the eye of the order of 18 mm. Advantageously, a distance between the lens and the eye greater than or equal to 15 mm in the conditions of use of the system is considered; this corresponds to a lens-implant distance greater than or equal to 19.43 mm; a lower limit of 19 mm is appropriate.

A reading object field is considered: a distance d₂ of 25 cm and an angle α of ±10° relative to the axis 2 can be chosen to define such a reading object field. This distance value is standard for patients with low vision. The choice of the angle α is representative of a customary reading field ensuring comfort when reading; this value corresponds to a range of 8 cm approximately on the page which allows for a few words to be seen on the page, i.e. the part of the text on which the reader is concentrating at a given instant. Another solution consists of using a field defined at the retina by an aperture angle of ±24°.

The system is considered operating in the region of a given wavelength in the visible spectrum, for example the central wavelength in the visible spectrum, i.e. 550 nm, but the reasoning and criteria described below could also be applied to any other wavelength in the visible spectrum. More precisely, the reasoning and criteria below are applied to a given wavelength in the visible spectrum. The reasoning and the criteria remain valid for other wavelengths of this spectrum. By contrast, due to the chromatic aberrations, the image spot over all of the wavelengths can be of a larger size than the size of the image spot for a given wavelength. In other words, the image spot in the violet has a size similar to the image spot in the red; however the position of these image spots on the retina can be slightly shifted, such that the image spot in the violet and in the red is larger than the respective sizes of the image spots in the violet and in the red. The reasoning and criteria therefore apply to any wavelength in the visible spectrum—but not necessarily to the image spot combining all of the wavelengths of the visible spectrum.

For point objects in the field thus defined and for a determined pupil size, the telescopic system produces an image spot on the back of the eye. If the ray tracing program marketed as Code V is used, the image spot is defined as twice the mean square deviation of the position of the light rays on the retina, for a ray bundle originating from a given point object and covering a pupil of a given size. Other methods of defining the image spot provide equivalent results and the use of this ray tracing program is not obligatory. It is also understood that the position of the implant in front of the pupil does not change the definition of the image spot.

According to the invention, for a pupil 1.5 mm in diameter, at the center of the object field, the image spot has a size greater than or equal to 20 μm. This value reflects the fact that it is not necessary, because of the poor visual acuity of patients with macular degeneration, for the image spot to be a point. A resolving power of 5 arc minutes, corresponding to an acuity of 2/10^{th}, produces an image spot of 24 μm on the retina; it is therefore not necessary, given the visual acuity of the patients, that the image spot is of a size markedly smaller than this value, because the final resolution is given by the retina.

For any point object in the reading object field—defined in the example in FIG. 2 by a distance d₂ of 25 cm and an aperture of ±10°—the image spot is of a size less than or equal to 50 μm. This higher value is chosen for the comfort of the patient. This image spot dimension prevents the patient perceiving a reduction in acuity. It is not necessary to measure the image spot for all of the possible positions of an object in the object field. For a revolution system, it is sufficient to choose three or four points on a radius (half-meridian); this solution remains valid for an aspherical system such as that given as an example below.

It is advantageous that the image spot always remains in this range of values, even in the case of the decentering of the lens 16 relative to its nominal position on the axis 2, in a range of at least ±0.5 mm. It is also advantageous for the image spot to always remain in this range of values, even in the case of angular displacement of the lens 16 relative to its nominal position, in a range of at least ±2°. These positioning tolerances are made possible by the choice of a non-nil image spot at the center of the object field.

The optical characteristics of the telescopic system are as follows. As explained above, the lens has a positive power. A power greater than or equal to 15 diopters is advantageous in order to ensure that the telescopic system has enlarging between 2 and 4. At least one of the faces of the lens can be aspherical. The implant has a central portion with a strong negative power; this power is typically less than −20 diopters, or even less than −60 diopters. These values, combined with the values proposed above for the distances d₁ and d₂, allow for a enlarging the telescopic system of between 2 and 4 to be obtained. A enlarging the telescopic system of between 2 and 4—preferably close to 3—for an object field in a range of ±10° is appropriate for patients with only slight macular degeneration. The system is simple to use and is discreet. It provides good comfort when reading with an appropriate reading speed.

The central portion of the implant typically has one or more of the following characteristics:

a diameter of between 1.5 and 2 mm; the lower value is sufficient for the contrast in the presence of the external lens to be greater than 0.25 for a 3 mm pupil; the higher value of the diameter range allows the patient to retain functional peripheral vision in the absence of the external lens;

an absolute value of power greater than or equal to 20 diopters; this value is chosen, taking account of the distances in the lens-eye system and the characteristics of the lens, in order to provide the required enlarging the telescopic system;

spherical surfaces; the absence of aspherical surfaces in the central portion of the implant facilitates the manufacture of the implant. This is possible because the optical performance of the desired system is not very high and is suited to the poor visual acuity of the patients;

a thickness at the center greater than or equal to 0.1 mm; this minimum value ensures the solidity of the implant;

a thickness at the edge lower than or equal to 0.5 mm and a total optical diameter of 5 to 6 mm; this maximum thickness value allows for correct implantation of the implant, while the value of the optical diameter ensures that the implant does not limit the entry of the rays into the eye.

The peripheral portion of the implant extends around the central zone. The total diameter of the implant is chosen so as to allow its positioning in the patient's eye, in front of the crystalline lens or an intrasaccular implant replacing the crystalline lens, or else in front of the pupil, as explained above. Typically, for a position behind the pupil, the implant has an external optical diameter of 5 to 6 mm, with, if appropriate, the haptics required for holding it in position in the patient's eye. The rear face of the central portion of the implant is advantageously concave with a radius comprised between 3 and 5 mm, preferentially a radius of 3.85 mm. This ensures that the telescopic system will be less sensitive to the decentering or angular displacement of the implant for a magnification 3 of the telescopic system. The central thickness of the implant and the radius of the front face of the central portion of the implant can advantageously be chosen (but this is not obligatory) as a function of any residual ametropia in the patient. If the patient has no residual ametropia, a radius of 4.40 mm and a thickness of 0.1 mm can be chosen for the implant. In this case, the peripheral portion of the implant has no optical effect and the patient's ametropia is corrected by the intrasaccular implant. The radius of the front surface of the implant can also be modified in order to correct the effects of residual ametropia in the patient over the optimum reading distance of the system. A choice of radii between 3.8 and 5.5 mm allows for correction of the effects of residual ametropia in the patient between −5 and +5 diopters, for a hydrophilic acrylic implant with an index of 1.460.

As for the central portion, it is advantageous that the peripheral portion of the implant is not aspherical, in order to facilitate the manufacture of the implant. This can be obtained by direct machining or moulding techniques or other techniques known per se for the manufacture of intraocular implants.

The lens external to the eye can have the following characteristics. The lens has a power greater than or equal to 15 diopters; this value is adjusted, taking account of the distance between the lens and the eye and taking account of the position of the reading object field, to provide a magnification between 2 and 4. The lens has a thickness at the center less than 15 mm. It is aspherical, which allows for the image spot sizes proposed in the considered reading object field to be retained; for example, for the front face of the lens a revolution surface can be used, the generator of which is a cone, for which the equation on one diameter can be written in the form Z=f(R) as follows:

$$Z = \frac{1}{{R}_{OSC}} [ \frac{{R}_{2}}{1 + \sqrt{1 - ( 1 + K ) {R}^{2} / {R}_{OSC}^{2}}} ]$$

with R, the distance from the point calculated to the optical axis; R_{OSC} the radius of curvature at the center and K the conicity or asphericity coefficient of the lens. For a lens made of a 1.665 index material, K can be chosen in the range [−1; 0] corresponding to an ellipse the shape of which varies between a sphere and a parabola, and preferably in the range [−0.6; −0.2], for example K=−0.42 as proposed below. These values are given as an example only because the value of K, allowing for the conditions on the image spot over all of a given object field according to the invention to be met, depends on the distances d₁ and d₂, the magnification chosen for the system and therefore the radii of curvature of the faces of the lenses, as well as (but to a lesser degree) on the position and the radii of curvature of the implant. It is obvious to a person skilled in the art that the asphericity can be pushed to a higher degree as far as required to allow the system to meet the conditions on the image spot; in this case, the higher order asphericity terms are added to the previous formula:

$$Z = \frac{1}{{R}_{OSC}} [ \frac{{R}^{2}}{1 + \sqrt{1 - ( 1 + K ) {R}^{2}} / {R}_{OSC}^{2}} ] + {\sum }_{i = 2}^{N MAX} {K}_{i} {R}^{25}$$

where NMAX is the degree of asphericity and the coefficients Kᵢ are the higher order asphericity coefficients.

The external lens can be tinted using filters commonly used in the correction of low vision in order to limit the glare effects commonly observed in people with ARMD, but this is not obligatory.

One example of a system according to the invention has the following characteristics. The enlarging the system is 3, for an implant corresponding to the eye model proposed above. The distance d₁ is 22.43 mm, which corresponds to a lens-eye distance of 18 mm, and the distance d₂ is 25 cm. The object field is defined by an angle α of ±10°. The lens is made of glass with an index of 1.665 and has a thickness at the center of 9.5 mm. The rear face is concave spherical with a radius of 250 mm. The front face has a radius of curvature at the center R_{osc} of 25.28 mm and an asphericity coefficient K of −0.42. With these characteristics, the lens has a power at the center of 24 diopters. The intraocular implant is of the type shown in FIG. 1 and is held behind the pupil and in front of an intrasaccular implant by haptics. It is biconcave spherical. The central portion of the rear face has a radius of 3.85 mm. The radius of the front face and the thickness of the central portion of the implant are given in the table below, as a function of the correction of ametropia produced by the peripheral portion of the implant.

| Residual | Central | Radius of the | Power of the |
|---|---|---|---|
| ametropia | thickness | front face | negative portion |
| (Diopters) | (mm) | (mm) | (Diopters) |
| 5 | 0.27 | −5.49 | −54.50 |
| 4.5 | 0.26 | −5.37 | −55.00 |
| 4 | 0.24 | −5.24 | −55.50 |
| 3.5 | 0.225 | −5.13 | −56.00 |
| 3 | 0.21 | −5.01 | −56.60 |
| 2.5 | 0.19 | −4.90 | −57.20 |
| 2 | 0.17 | −4.79 | −57.70 |
| 1.5 | 0.15 | −4.70 | −58.20 |
| 1 | 0.13 | −4.59 | −58.80 |
| 0.5 | 0.11 | −4.49 | −59.40 |
| 0 | 0.1 | −4.40 | −60.00 |
| −0.5 | 0.26 | −4.54 | −59.20 |
| −1 | 0.24 | −4.44 | −59.80 |
| −1.5 | 0.225 | −4.36 | −60.30 |
| −2 | 0.205 | −4.27 | −60.90 |
| −2.5 | 0.185 | −4.18 | −61.50 |
| −3 | 0.17 | −4.11 | −62.00 |
| −3.5 | 0.15 | −4.03 | −62.60 |
| −4 | 0.135 | −3.95 | −63.20 |
| −4.5 | 0.115 | −3.88 | −63.70 |
| −5 | 0.1 | −3.81 | −64.30 |

The central portion of the implant extends over a diameter of 1.9 mm.

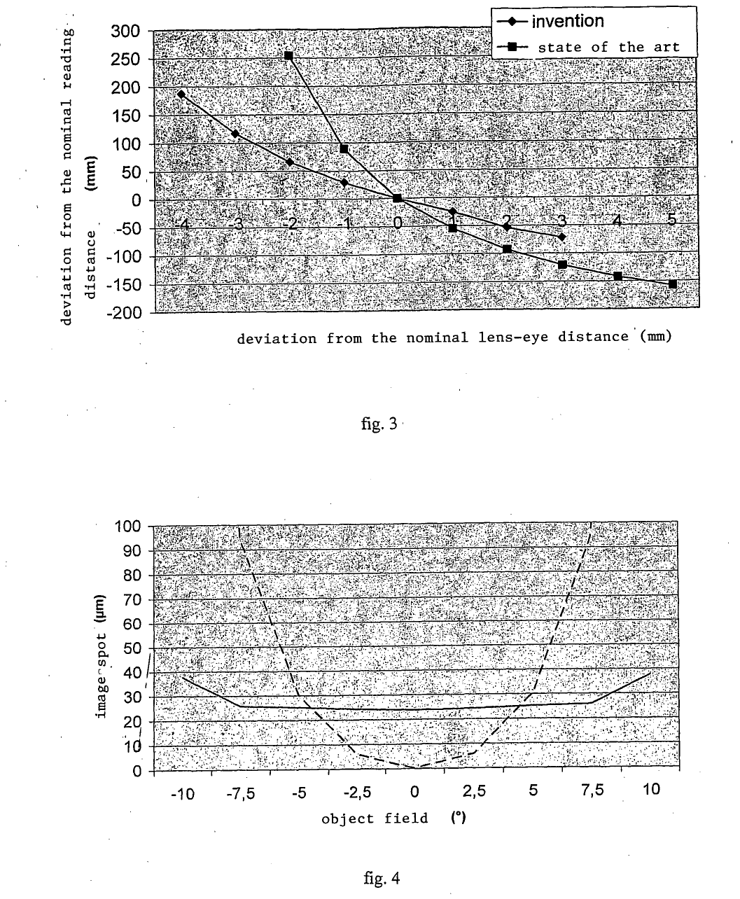

FIG. 3 to 6 show the optical characteristics of the example discussed, for an implant without correction of ametropia. FIG. 3 is a diagram of the reading distance in mm, as a function of the lens-eye distance in mm, in a system according to the invention and in a system according to the state of the art represented by U.S. Pat. No. 4,957,506. As indicated above, the system in the example is envisaged for a nominal lens-eye distance of 18 mm; for this lens-eye distance, the reading field is situated at a distance d₂ of 25 cm relative to the front face. The graph in FIG. 3 shows the necessary variations in the distance d₂ in order for the system to retain the same optical properties, as a function of the variations in the lens-eye distance. The figure shows that the reading distance of the system according to the invention remains comprised between 18 and 43 cm (deviation of −7 cm to +18 cm), when the lens-eye distance varies between 14 and 21 mm (deviation of −4 mm to +3 mm). In other words, even when the position of the lens along the axis 2 deviates from the nominal position, the system of the invention can still be used. By way of comparison, the graph in FIG. 3 shows the values calculated for a system according to U.S. Pat. No. 4,957,506; the graph shows that this system of the state of the art is much more sensitive to the position of the lens in front of the eye.

FIGS. 4 to 6 are diagrams showing the characteristics of the example proposed, compared to the state of the art disclosed in U.S. Pat. No. 4,957,506, in the table in column 5. FIG. 4 gives the size of the image spot in the object field, as a function of the angle α in degrees. Specifically, for each angle value plotted on the x-axis, a point of the object field was considered and the size of the image spot is shown on the graph in μm. The figure shows the values obtained in the system of the invention with a thick line and the values of the state of the art with a dotted line. It can be seen that the image spot has a size comprised between 20 and 40 μm for all of the points of the object field in the system of the invention. By contrast, in the magnification system of the state of the art, the size of the image spot at the center is nil. The size of the image spot exceeds 40 μm for an angle value of the order of 50 and exceeds 100 μm for an angle value of the order of 7.5°. In other words, near the axis, the system of the state of the art is too effective relative to the acuity of the wearer; moving away from the axis, the performance of the system decreases rapidly and the reading field is therefore narrow. The invention, by allowing a reduction in the optical performance on the axis, ensures a wider field of vision.

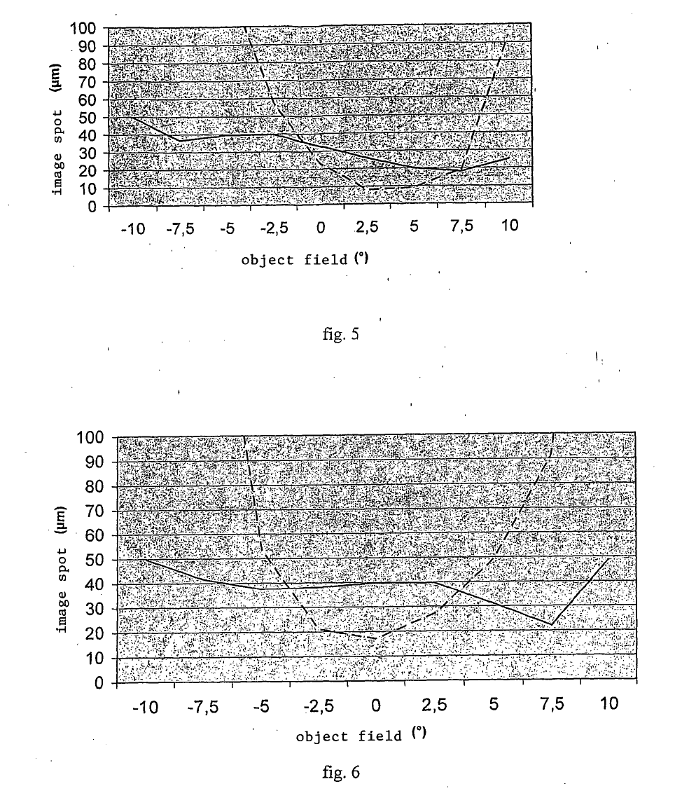

FIGS. 5 and 6 illustrate the effect of the incorrect positioning of the lens, relative to the nominal position. FIG. 5 is similar to FIG. 4, but the lens is off-center relative to the axis, by a distance of 1 mm. The figure shows that the size of the image spot of the system of the invention is still comprised between 20 and 50 μm over the entire object field. The system of the state of the art has an image spot size that greatly exceeds 70 μm on either side of the optical axis. In other words, in the system of the invention, the decentering of the lens does not cause any loss of optical performance in the field of vision when reading; by contrast, in the system of the state of the art, a decentering of 1 mm causes a reduction of more than a third of the amplitude of the field of vision.

FIG. 6 is similar to FIG. 4, but the lens is rotated relative to the axis, by an angle of 5°. The figure shows that the image spot size of the system of the invention is still comprised between 20 and 50 μm over the entire object field. The system of the state of the art has an image spot size that exceeds 100 μm over the object field, on either side of the optical axis. As for the decentering, a rotation of the lens in the system of the invention does not lead to any loss of optical performance in the field of vision when reading; by contrast, in the system of the state of the art, a 5° rotation of the lens leads to a reduction of close to a quarter of the amplitude of the field of vision.

In the example in the figures, a range of variation of the angular position of ±5° and a range of decentering of ±1 mm were considered; these values are higher than the respective values of ±2° and +0.5 mm proposed above. The example shows that it is possible to set a limit on the size of the image spot for larger variations of the position of the lens, while retaining a suitable system for the wearer. Respective ranges of ±10° and mm can also be used in order to allow even larger variations in the mounting conditions.

The invention therefore allows a wider field of vision to be obtained, as shown by FIG. 4. Moreover, it provides a system of retinal magnification that is not very sensitive to the variations of the position of the external lens, relative to the nominal position.

One example of the system according to the invention has been given, as well as ranges of values of the different characteristics of the system. Other embodiments of the invention can be obtained by optimization of the surfaces of the lens and the implant. The optimization can be carried out in a manner known per se, using software such as that marketed under the trade mark Code V by the company ORA (Optical Research Associates). The optimization can be carried out as follows:

a standard eye model is chosen, or, for a customized definition, the characteristics of the wearer's eye are determined;

wearing conditions of the lens are chosen, either for a standard wearer, or customized for a given wearer;

a rear face of an implant and a lens is chosen, for example with the values proposed above;

a starting thickness and front face are chosen for the lens and the implant, in order to ensure a reasonable image spot on the axis and the desired magnification and reading distance d₁;

limits are set on the system, corresponding to the desired magnification and reading distance d₁;

limits are set, corresponding to image spot sizes for several points distributed in the object field;

the shape and the thickness of the front faces of the lens and the implant are varied in order to approach the targets.

It is also possible to set limits representative of incorrect positioning of the lens. For example, the image spot sizes for a lens off-center by 1 mm and for a lens rotated by 5° can be limited.

In the example, the front faces of the lens and the implant are optimized. Other faces can be optimized for example the front and rear faces of the lens can be optimized simultaneously. Optimization can be carried out in order to take account of a correction of ametropia by the peripheral portion of the implant, simply by modifying the standard eye model so that it represents the required correction of ametropia.

Such optimization makes it possible to obtain embodiments of systems according to the invention, for other eye models or other wearing conditions than those proposed in the example.

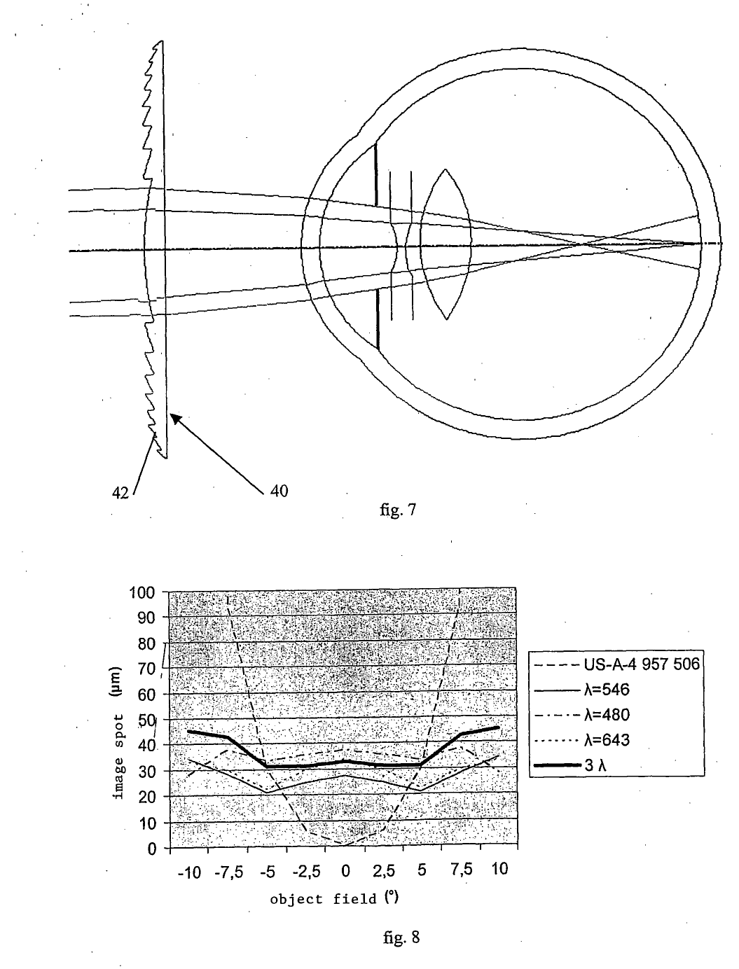

FIG. 7 shows a view similar to that in FIG. 1, for another embodiment of the invention. The system in FIG. 7 differs from that in FIG. 1 in that the lens 40 is a Fresnel lens. The front face 42 of the lens therefore has the standard shape of a Fresnel lens, with concentric zones. The solution in FIG. 7 allows for the thickness of the lens to be limited: compared to the example proposed above of a lens with a thickness at the center of 9.5 mm, the solution in FIG. 7 allows for the same power of 24 diopters to be provided at the center, with a thickness of the order of 2 mm. The same material and the same asphericity of the front face are retained. The radii of the Fresnel lens can be determined in a manner known per se; for example the following radii can be considered:

thickness at the center of the Fresnel lens: 2 mm

step value: 1 mm.

With this example, the focal size values described with reference to FIGS. 1 to 6 are retained.

It is also possible, in combination or alternating with the Fresnel lens shown in FIG. 207, to consider a material with a lower index and with a higher Abbe number than in the example of FIG. 1. This solution allows for the chromatism of the system to be reduced. As an example, the material of the lens in FIG. 1 has an index of 1.665 and an Abbe number of 31. For a given wavelength, the image spot size is comprised between 20 and 50 μm, as explained above. However, when all of the wavelengths of the visible spectrum are considered, the size of the image spot for a point of the object space can reach 300 μm, in particular at the edge of the field.

Instead of this material a material with an index of 1.502 and with an Abbe number of 58, such as the material sold under the name CR39 by PPG Industries, Pittsburgh, USA, can be used. In this case, the property of an image spot is kept at between 20 and 50 μm for a wavelength; however, the size of the image spot for a point of the object space, over all of the wavelengths of the visible spectrum, is then less than 150 μm, which significantly reduces the interference related to the chromatism of the system.

It is also possible to envisage, in the embodiment in FIG. 1 or in the embodiment in FIG. 7, that the lens has diffractive properties. The lens then has surface and/or index variations close to the wavelengths transmitted.

As an example, it is possible to provide circular concentric zones on the front face of the lens, similar to those shown in FIG. 7, but with a step with a size of a different order of magnitude. For example, a calculation of the diffractive properties of the lens for a central wavelength in the visible spectrum can be considered, in the range of 500 to 600 nm, such as λ=546 nm. For this wavelength, in the example of the lens in FIG. 1, it is possible to choose a step of the order of:
(n−1).λ=0.665*0.546=0.366 μm
where n is the refractive index of the material of the lens. It is thus possible to provide one or more diffractive surfaces on the lens. Such diffractive properties allow for the chromatism of the system to be limited.

These diffractive properties advantageously have a rotational symmetry, like the rest of the magnification system. The system as a whole thus has a rotational symmetry, which prevents the favoring of one portion of the field of vision.

It is possible for example to use a diffractive element, the properties of which are realized by modification of the profile of the surface, known as a kinoform phase plate. This element can be applied or provided on the front face or on the rear face of the lens in FIG. 1, or on the rear face of the lens in FIG. 7.

Below is an example in a configuration similar to that in FIG. 1. The lens is made from a material with an index of 1.665 and with an Abbe number of 31, as in the example in FIG. 1. The front face 18 is aspherical and has a radius of curvature at the center R_{osc} of 26.731 mm, an asphericity coefficient K=−0.734 and a 1^{st} higher order asphericity coefficient K₁=4.95e-006 mm⁻¹. The rear face 20 is concave spherical with a radius of 150 mm. The thickness at the center is 9.5 mm.

The diffractive portion is formed by a phase filter, providing a phase shift in the form:

$$Φ ( r ) = 2 π / λ x ( {\sum }_{i} {C}_{1} {r}^{2 i} )$$

where

r, distance from the point to the optical axis in mm.

λ=546.1 nm, reference wavelength.

C₁=−0.00151 mm⁻¹

C₂=2.516e-6 mm⁻³

C₃=−1.46e-8 mm⁻⁵

C₄=3.75e-11 mm⁻⁷ and

—C₅=−2.84e-14 mm⁻⁹

This phase shift can in particular be carried out by a kinoform phase plate.

As in the example in FIG. 1, the implant is biconcave spherical, with a rear face with a radius of 3.85 mm; the radius of the front face and the thickness at the center of the implant depend on the corrected ametropia. For zero ametropia, a front face with a radius of 4.986 mm and a thickness at the center of 0.1 mm is considered for example.

In these conditions, the system has, for any wavelength in the visible spectrum, a focal spot size less than 50 μm for any point object in the reading object field. When all of the wavelengths in the visible spectrum are considered, a focal spot much smaller than the dimension of 300 μm mentioned above is obtained for any point object in the reading object field.

For greater simplicity, it is possible to consider only three values of wavelengths, distributed in the visible spectrum. For example, the following are considered:

a wavelength in the blue, between 400 and 500 nm

a central wavelength, between 500 and 600 nm and

a wavelength in the red, between 600 and 800 nm.

The consideration of three wavelengths thus distributed is sufficient to obtain focal spot sizes representative of those obtained considering all of the wavelengths of the visible spectrum.

Typically, for the focal spot of a point object in the reading object field for three wavelengths thus chosen, a size of 20 to 50 μm is thus obtained. In the following examples it will be noted that the size of the focal spot obtained for three wavelengths is calculated, as proposed above, using the mean square deviation. As a result, the value of the focal spot for three wavelengths is not a simple function of the three focal spot values for the three wavelengths considered.

As an example, wavelengths of λ₃=643.8 nm, λ₂=546.1 nm and λ₁=480 nm are considered. FIG. 8 shows a graph similar to that in FIG. 4, giving the focal spot sizes for the wavelengths λ₁, λ₂ and λ₃ for these three wavelengths as well as the focal spot size in the system of the state of the art described in patent U.S. Pat. No. 4,957,506, for a wavelength of 546.1 nm. It can be seen, as was the case in FIG. 4, that the focal spot size is still comprised between 20 and 50 μm for each of the wavelengths, but also when the light at the three lengths in question is considered. By way of comparison, the focal spot size in the system of the state of the art is small on the axis—where the patient has lost vision—but very large on the periphery of the object field.

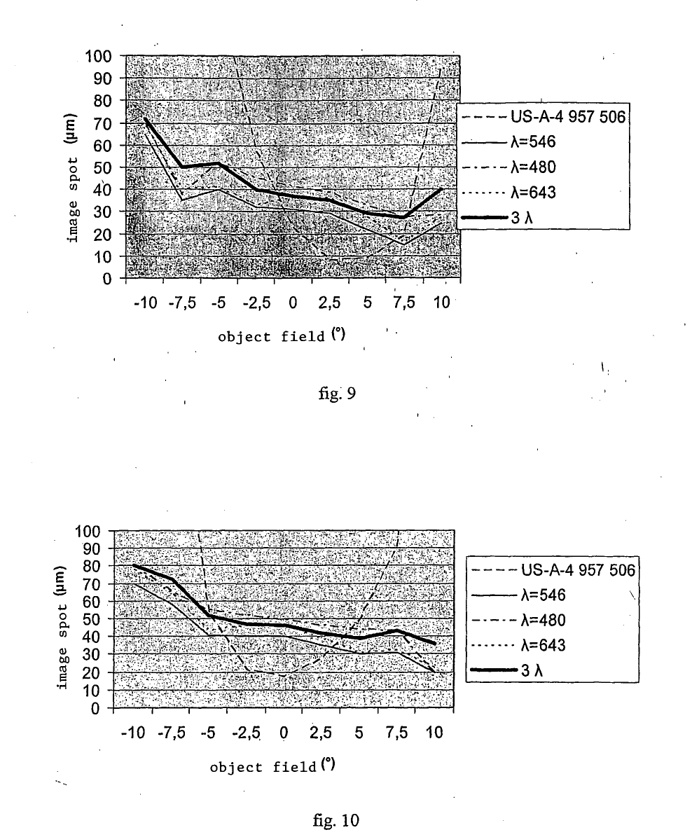

FIG. 9 shows a graph similar to that in FIG. 5, giving the focal spot sizes for the wavelengths λ₁, λ₂ and λ₃, for these three wavelengths as well as the focal spot size in the system of the state of the art described in patent U.S. Pat. No. 4,957,506. FIG. 9 shows the example of a decentering of the lens by 1 mm. It can be seen on the graph that the focal spot size is still comprised between 5 and 80 μm, for each of the wavelengths considered and for the light at these three wavelengths.

FIG. 10 shows a graph similar to that in FIG. 6, giving the focal spot sizes for the wavelengths λ₁, λ₂ and λ₃, for these three wavelengths as well as the focal spot size in the system of the state of the art described in patent U.S. Pat. No. 4,957,506. FIG. 10 shows the example of an angular displacement of the lens of 5°. As in the example of FIG. 9, it can be seen on the graph that the focal point size is still comprised between 5 and 80 μm, for each of the wavelengths considered and for the light at these three wavelengths.

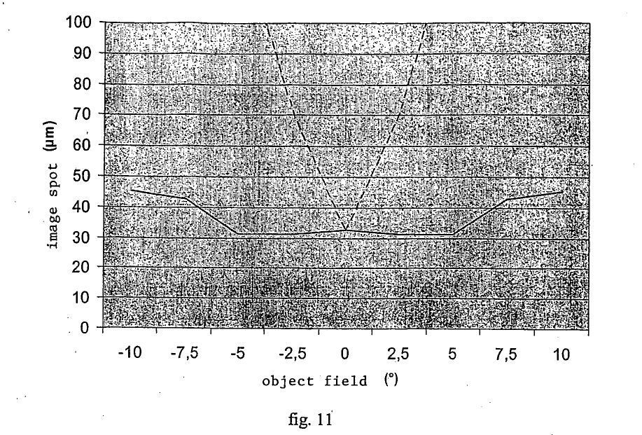

Finally, FIG. 11 is a graph similar to that of FIG. 8; the graph shows with a thick line the focal spot size calculated for the three wavelengths λ₁, λ₂, λ₃, in the system with diffractive properties given as an example. The graph also shows with dotted lines the focal spot size calculated for these three wavelengths in the system in document U.S. Pat. No. 4,957,506. A comparison of FIG. 8 and FIG. 11 shows that the focal spot size in the system of the state of the art increases even more rapidly when, instead of a single wavelength, several wavelengths distributed in the spectrum, are observed.

A graph similar to that in FIG. 3, for several wavelengths, has not been shown. Results very similar to those represented in FIG. 3 are obtained, and the variations depend only to a small degree or not at all on the wavelength.

The diffractive properties of the lens can be determined by optimization, according to the principles described above. It is possible to firstly optimize the lens and the implant, without particular diffractive properties, in order to obtain a system close to the desired solution, and then optimize the system again, integrating the diffractive properties. In this way, the properties of the lens obtained initially are significantly modified. Alternatively, it is possible to optimize the lens by integrating the diffractive properties from the start.

Of course, the invention is not limited to the preferred examples given above. Other wearing conditions than those proposed as an example could be used; another eye model could be used. It is also possible to use other methods of optimization than those proposed.

What is claimed is:

1. A system for enlarging a retinal image, comprising:
an intraocular implant having a peripheral portion and a central portion with negative power,
a lens with positive power designed to be arranged outside the eye, the lens and the implant being able to produce an enlarged image of an object at the back of an eye of a standard user,
in which, for a pupil 1.5 mm in diameter, any point object in a reading object field produces at the back of the eye an image spot of a size comprised between 20 and 50 μm, for a wavelength in the visible spectrum.

2. The system in claim 1, wherein, when an angular position of the lens varies in a range of ±2°relative to its nominal position, for a pupil 1.5 mm in diameter, any point object in a reading object field produces at the back of the eye an image spot of a size comprised between 20 and 50 μm, for a wavelength in the visible spectrum.

3. The system in claim 1, wherein, when an angular position of the lens varies in a range of ±5°, preferably ±10° relative to its nominal position, for a pupil 1.5 mm in diameter, any point object in a reading object field produces at the back of the eye an image spot of a size comprised between 20 and 50 μm, for a wavelength in the visible spectrum.

4. The system in claim 1, 2 or 3, wherein, when a decentering of the lens varies in a range of ±0.2 mm relative to a nominal position, any point object in a reading object field produces at the back of the eye an image spot of a size comprised between 20 and 50 μm, for a wavelength in the visible spectrum.

5. The system in claim 1, 2 or 3, wherein, when a decentering of the lens varies in a range of ±1 mm, preferably ±2 mm, relative to a nominal position, any point object in a reading object field produces at the back of the eye an image spot of a size comprised between 20 and 50 μm, for a wavelength in the visible spectrum.

6. The system of claim 1, wherein the lens has diffractive properties.

7. The system in claim 6, wherein the diffractive properties are obtained by modification of a profile of one of the surfaces of the lens.

8. The system in claim 6, wherein, when a decentering of the lens varies in a range of ±1 mm, relative to a nominal position, any point object in a reading object field produces at the back of the eye an image spot of a size comprised between 5 and 80 μm, for three wavelengths distributed in the visible spectrum.

9. The system in claim 6, wherein, when an angular position of the lens varies in a range of ±5° relative to its nominal position, for a pupil 1.5 mm in diameter, any point object in a reading object field produces at the back of the eye an image spot of a size comprised between 5 and 80 μm, for three wavelengths distributed in the visible spectrum.

10. The system in claim 8 or 9, wherein the three wavelengths are respectively chosen in the ranges of 400 to 500 nm, 500 to 600 nm and 600 to 800 nm.

11. The system of claim 1, wherein the central portion of the implant is spherical.

12. The system of claim 1, wherein the front face of the lens is a cone the conicity of which is comprised between 0 and −1, preferably comprised between −0.2 and −0.6.

13. The system of claim 1, wherein the lens is a Fresnel lens.

14. The system of claim 1, wherein it has an enlargement comprised between 2 and 4.

15. The system of claim 1, having, in use, a distance between the lens and the implant greater than or equal to 19 mm.

16. The system of claim 1, wherein the reading object field is situated at a distance (d₂) of 25 cm from the lens and covers an angle (α) of 10°.

17. The system of claim 1, wherein the reading object field is defined by an aperture angle at the retina of ±24°.

18. A method for determination by optimization of a system for enlarging a retinal image, comprising:
choosing an eye model, wearing conditions, an intraocular implant and a lens external to the eye;
modifying the characteristics of the implant and the lens in order that, in a reading object field, any point object produces at the back of the eye an image spot of a size comprised between 20 and 50 μm.

19. The method in claim 18, wherein the modification stage is also carried out in order that, in the presence of a variation of the angular position of the lens relative to the chosen wearing conditions, in a range of ±2°, any point object in a reading object field produces at the back of the eye an image spot of a size comprised between 20 and 50 μm, for a wavelength in the visible spectrum.

20. The method in claim 18, wherein the modification stage is also carried out in order that, in the presence of a variation of the angular position of the lens relative to the wearing conditions chosen, in a range of ±5°, preferably in a range of ±10°, any point object in a reading object field produces at the back of the eye an image spot of a size comprised between 20 and 50 μm, for a wavelength in the visible spectrum.

21. The method in one of claims 18 to 20, wherein the modification stage is also carried out in order that, in the presence of a decentering of the lens in a range of ±0.5 mm relative to the wearing conditions chosen, any point object in a reading object field produces at the back of the eye an image spot of a size comprised between 20 and 50 μm, for a wavelength in the visible spectrum.

22. The method in one of claims 18 to 20, wherein the modification stage is also carried out in order that, in the presence of a decentering of the lens in a range of ±1 mm, preferably ±2 mm, relative to the wearing conditions chosen, any point object in a reading object field produces at the back of the eye an image spot of a size comprised between 20 and 50 μm, for a wavelength in the visible spectrum.

23. The method of claim 18, wherein the modification stage comprises the application of diffractive properties to the lens.

24. The method in claim 23, wherein the modification stage is also carried out in order that, in the presence of a variation of an angular position of the lens relative to the wearing conditions chosen, in a range of ±5°, any point object in a reading object field produces at the back of the eye an image spot of a size comprised between 5 and 80 μm, for three wavelengths distributed in the visible spectrum.

25. The method in claim 23 or 24, wherein the modification stage is also carried out in order that in the presence of a decentering of the lens in a range of ±1 mm relative to the wearing conditions chosen, any point object in a reading object field produces at the back of the eye an image spot of a size comprised between 5 and 80 μm, for three wavelengths distributed in the visible spectrum.

26. The method of claim 18, wherein the reading object field is situated at a distance (d₂) of 25 cm from the lens and covers an angle (α) of 10°.

27. The method of claim 18, wherein the reading object field is defined by an aperture angle at the retina of ±24°.
